(19) United States
(12) Patent Application Publication
(10) Pub. No.: US 2009/0253918 A1
(43) Pub. Date: Oct. 8, 2009

(54) NOVEL INTERMEDIATE FOR GLYT1 INHIBITOR

(75) Inventors: Ioannis Nicolaos Houpis, Beerse (BE); Didier Philippe Robert Schils, Beerse (BE)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: JANSSEN PHARMACEUTICALS, N.V., Beerse, BE (BE)

(21) Appl. No.: 12/444,010

(22) PCT Filed: Sep. 26, 2007
(86) PCT No.: PCT/EP07/60203

§ 371 Date: Apr. 2, 2009

(30) Foreign Application Priority Data
Oct. 2, 2006 (EP) 06121643.8

(51) Int. Cl. C07D 409/10

(52) U.S. Cl. 549/60

(57) ABSTRACT

This invention relates to an improved process for preparing the glycine transport 1 (GlyT1) inhibitor (Z)-N-(1-(4-(2-furyl)phenyl)-1-(3-thienyl-prop-1-en-3-yl) sarcosine via the novel intermediate (Z)-3-(4-(2-furyl)phenyl)-3-(3-thienyl)-prop-2-en-1-ol and the preparation of the latter.

### FIELD OF THE INVENTION

This invention relates to an improved process for preparing the glycine transport 1 (GlyT1) inhibitor N-[(2Z)-3-[4-(2-furyl)phenyl]-3-(3-thienyl)-2-propen-1-yl]-N-methylglycine via the novel intermediate (Z)-3-(4-(2-furyl)phenyl)-3-(3-thienyl)-2-propen-1-ol and the preparation of the latter.

### BACKGROUND OF THE INVENTION

The glycine transport 1 (GlyT1) inhibitor N-[(2Z)-3-[4-(2-furyl)phenyl]-3-(3-thienyl)-2-propen-1-yl]-N-methylglycine of formula (II-a) and its pharmaceutically acceptable salts of formula (II-b) are disclosed in WO-02/066456. In the cited patent application, compounds like (II-a) are depicted as uncharged, neutral compounds. Physically such compounds occur predominantly as charged compounds called zwitterions or dipolar ions and hereinunder the compound of formula (II-a) is represented as a zwitterion.

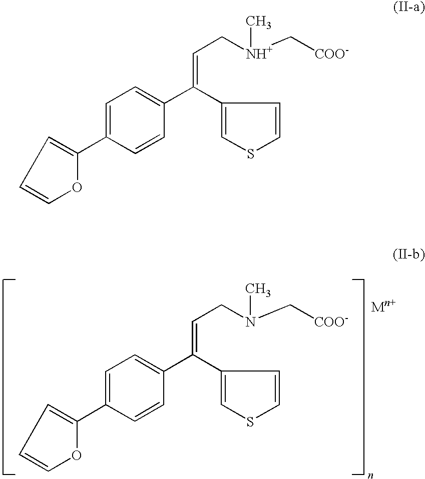

The preparation of compounds (II-a) and (II-b) [Ar₁=3-thienyl; Ar₂=2-furyl] is generically disclosed in Scheme 1 of WO-02/066456 which is reproduced hereunder in amended form.

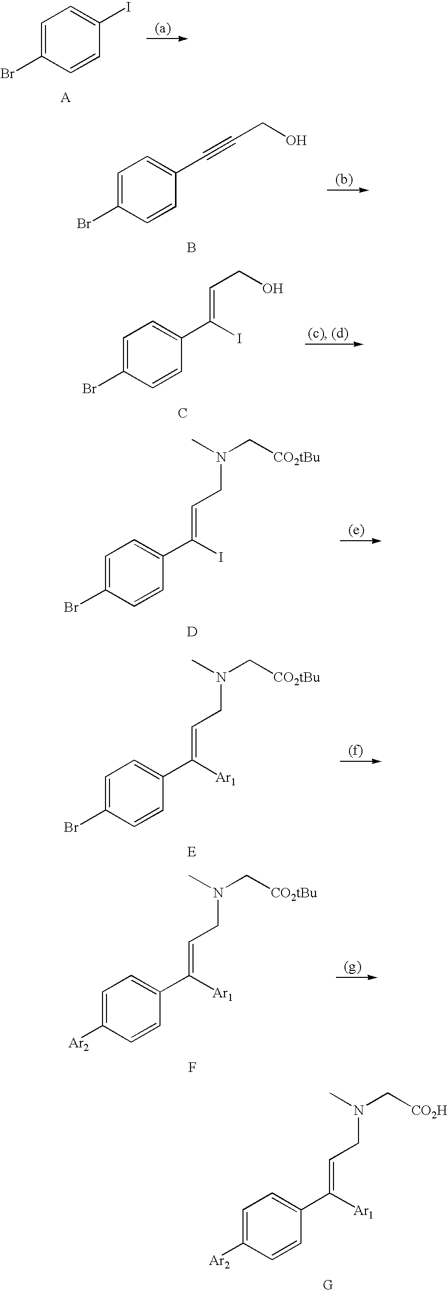

(a): propargyl alcohol, Pd(PPh₃)₄, CuI, Et₃N, r.t., overnight; (b): Red-Al, THF, 0° C., 1 hour, then EtOAc, 12, −78° C. to r.t., overnight; (c): NBS, PPh₃, CH₂Cl₂, −40° C., 1 hour, (d): t-butylsarcosine, K₂CO₃, KI, MeCN, r.t., overnight; (e): Ar₁B(OH)₂, Pd(PPh₃)₄, 2M Na₂CO₃, DME, 90° C., 4.5 hours; (f): Ar₂B(OH)₂, Pd(PPh₃)₄, 2M Na₂CO₃, DME, reflux, 3 hours; (g): formic acid, 40° C., overnight.

The strategy of the above synthetic route allows one to create a library of chemically diverse compounds using common intermediates. Whilst this is a suitable approach when screening for particular activities in multiple compounds, it is not suitable for the purpose of preparing pharmaceutical grade products such as the compounds (II-a) and (II-b) for a couple of reasons. The fact that two Pd catalyzed coupling reactions occur near the end of the route results in a Pd contaminated product that is difficult to purify without compromising yield. In addition, the first of the Pd catalyzed coupling reactions (step e) is not entirely selective resulting in the presence of the bis-thienyl impurity H in the end product.

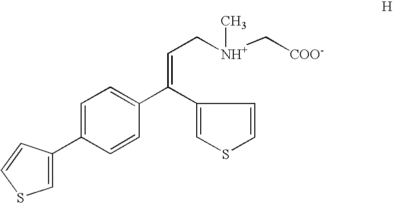

This invention relates to an improved process for preparing the glycine transport 1 (GlyT1) inhibitor N-[(2Z)-3-[4-(2-furyl)phenyl]-3-(3-thienyl)-2-propen-1-yl]-N-methylglycine via the novel intermediate (Z)-3-(4-(2-furyl)phenyl)-3-(3-thienyl)-2-propen-1-ol and the preparation of the latter compound.

The novel intermediate is converted into the compounds (II-a) and (II-b) by conversion of the hydroxyl group into a leaving group and reacting the thus obtained intermediate with methyl N-methylglycinate

The aforementioned intermediates are novel and have the formula (I)

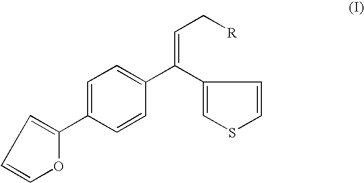

wherein R represents hydroxyl, or a leaving group L selected from the group consisting of chloro, bromo, iodo, methanesulfonyl, ethanesulfonyl, trifluoromethanesulfonyl, benzenesulfonyl, 4-methylbenzenesulfonyl, bromobenzenesulfonyl, and phosphonates.

Of particular interest is the compound wherein R represents hydroxyl and has the formula (I-a)

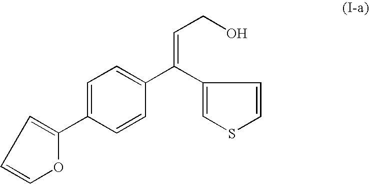

The invention further concerns a process of converting a compound of formula (I-a) as defined hereinbefore into a compound of formula (II-a) or a pharmaceutically acceptable salt (II-b) thereof,

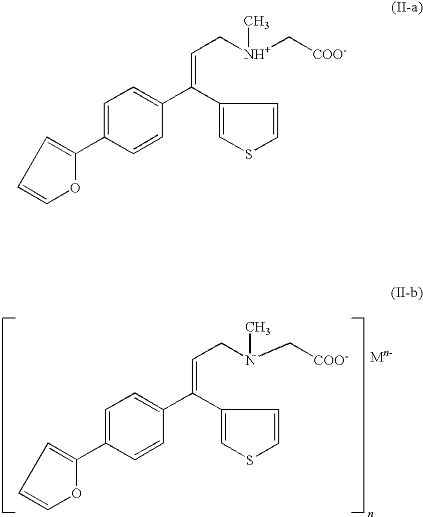

wherein n represents 1 or 2, and
Mⁿ⁺ represents a n-valent metal ion selected from the group consisting of the monovalent metal ions Li⁺, Na⁺, K⁺ and the divalent metal ions Mg²⁺ and Ca²⁺, comprising the steps of
(a) reacting the compound of (I-a) with a sulfonyl halide in a tertiary amine thus forming a compound of (I-b)

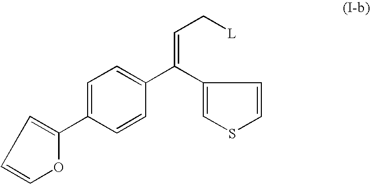

wherein L represents a leaving group;
(b) treating the compound of formula (I-b) in situ with sarcosine methyl ester hydrochloride thus forming a compound of formula (III)

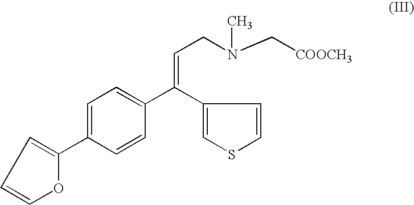

(c) hydrolyzing the compound of formula (III) in the presence of a base (Mⁿ⁺)(OH⁻)ₙ or (Mⁿ⁺)_{n/2}(CO₃²⁻) in a solvent to yield a compound of formula (II-b)

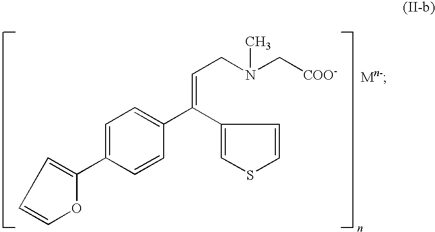

and
(d) optionally converting the thus obtained salt form (II-b) into the compound of formula (II-a)

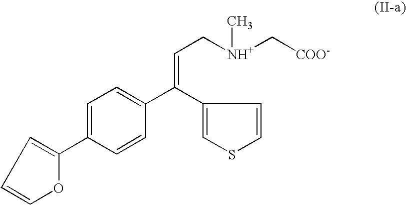

by treatment with an acid in an appropriate solvent.

This invention further concerns a process of preparing the compound of formula (I-a) comprising the steps of
(a) coupling an intermediate of formula (IV) with 2-propyn-1-ol (V) in the presence of a Pd catalyst in an appropriate solvent to yield an intermediate of formula (VI)

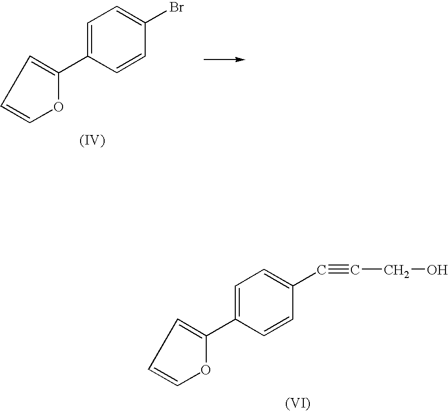

(b) converting the intermediate of formula (VI) by reaction with sodium bis(2-methoxyethoxy)aluminum hydride, followed by treatment in situ with iodine into an intermediate of formula (VII)

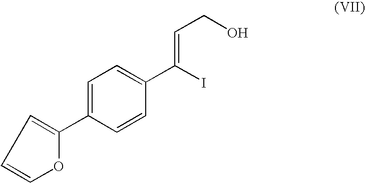

(c) coupling the intermediate of formula (VII) with 3-thienylboronic acid (VIII) in the presence of Pd/C and triphenyl phosphine, thus yielding a compound of formula (I-a).

Further, this invention also concerns a process of preparing the compound of formula (I-a) comprising the steps of
(a) coupling an intermediate of formula (IV) with 2-propyn-1-ol (V) in the presence of a Pd catalyst in an appropriate solvent to yield an intermediate of formula (VI)

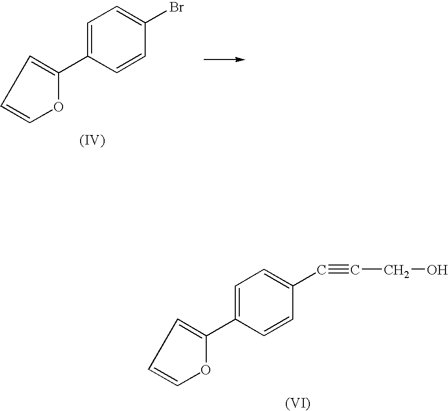

(b) converting the intermediate of formula (VI) by reaction with sodium bis(2-methoxyethoxy)aluminum hydride into an intermediate of formula (IX)

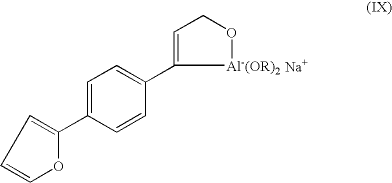

wherein each OR represents methoxyethoxy;
(c) coupling the intermediate of formula (IX) with 3-bromothiophene (X) in the presence of [1,3-bis(2,6-diisopropylphenyl)imidazol-2-ylidene](3-chloropyridyl palladium(II) dichloride (PEPPSI™ IPr) and zinc chloride, thus yielding a compound of formula (I-a).

### EXPERIMENTAL PART

### A. Preparation of the Intermediate Compounds

### Example A1

### Preparation of Intermediate 1

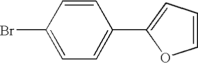

A 10 L vessel under N₂ was charged with 4-bromoiodobenzene (301 g; 1.06 moles), 2-furanboronic acid (148.81 g; 1.25 moles), triphenylphosphine (13.95 g; 0.05 moles), tetra-n-butylammonium bromide (377.29 g; 1.1 moles), sodium carbonate (225.53 g; 2 moles), palladium on carbon 10% (60.20 g; 56.57 mmoles), tetrahydrofuran (3.72 L) and water (3.72 L). The reaction mixture was heated at 60° C. for 20 until gas chromatography showed no unreacted starting material. After cooling to room temperature, the reaction mixture was filtered over decalite. The organic layer was separated and washed with a saturated NaCl solution (2 L), concentrated under reduced pressure and diluted with toluene (1.5 L). The solids which formed were filtered off and washed with toluene (1 L). The combined toluene layers were concentrated and purified by column chromatography (silica gel; hexane). The product was crystallized from ethanol/water (2.27 L/2.27 L) and dried. Yield: 266 g of intermediate 1 (2-(4-bromophenyl)-furan) (94%).

### Example A2

### Preparation of Intermediate 2

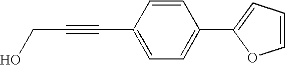

A 350 mL flask equipped with a mechanical stirrer was charged with N-methyl-pyrrolidone (100 ml; 1.04 moles) and degassed during 15 minutes under nitrogen. Reagents were added in the following order: 2-(4-bromophenyl)-furan (int. 1; 22.31 g; 0.1 moles); copper(I)iodide (761.8 mg, 4 mmoles); 1-butanamine (11.88 mL, 120 mmoles) and tetrakis(triphenylphosphine)palladium (2.31 g, 2 mmoles). The reaction mixture was heated to 50° C. and a solution of 2-propyn-1-ol (6.99 mL, 120 mmoles) in N-methylpyrrolidone (10 mL) was added slowly over 1 h (syringe pump: 17 ml/h). The reaction mixture was transferred to a separation funnel and treated with water (400 ml) and isopropyl acetate (100 mL). The water layer was extracted a second time with isopropyl acetate (100 mL). The combined organic layers were washed twice with an ammonia solution (20 mL saturated ammonia diluted in 100 ml water). The organic layer was concentrated in vacuo to afford 16 g of crude product which can further purified by chromatography (silica gel; dichloromethane). Yield: Intermediate 2 (3-(4-(2-furyl)phenyl)-2-propyn-1-ol)

¹H NMR (400 MHz, CHLOROFORM-d) δ ppm 2.00 (s, 1H) 4.48 (s, 2H) 6.44 (dd, J=3.27, 1.76 Hz, 1H) 6.63 (d, J=3.27 Hz, 1H) 7.42 (ddd, J=8.31, 1.51, 1.26 Hz, 2H) 7.44 (d, J=1.51 Hz, 1H) 7.57 (dt, J=8.37, 1.73 Hz, 2H)

¹³C NMR (101 MHz, CHLOROFORM-d) δ ppm 51.57 (1 C) 85.69 (1 C) 88.08 (1 C) 105.93 (1 C) 111.76 (1 C) 121.32 (1 C) 123.59 (2 C) 130.83 (1 C) 132.03 (2 C) 142.49 (1 C) 153.36 (1 C)

### Example A3

### a. Preparation of Intermediate 3

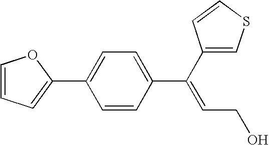

A round bottom flask was charged with intermediate 2 (6.80 g, 34.31 mmoles) and degassed tetrahydrofuran (55 mL) and cooled to 0° C. Sodium bis(2-methoxyethoxy) aluminum hydride (9.01 g, 44.59 mmoles) was added without control of internal temperature. The mixture was cooled to −20° C., stirred for 30 minutes and treated with a solution of iodine (10.45 g, 41.17 mmoles) in tetrahydrofuran (25 mL). After stirring for 30 minutes the reaction was quenched by addition of a sodium hydrogen sulfite solution (39%, 120 mL), water (50 mL) and the pH was adjusted to about 2-3 with concentrated HCl. Tetrahydrofuran (60-70 mL) was added to facilitate phase separation. The organic layer was washed twice with sodium carbonate and the volume was reduced to 70 mL. This solution was degassed with N₂, as well as water (70 mL). The two degassed liquids were mixed and potassium carbonate (10 g, 72.36 mmoles), 3-thiopheneboronic acid (5 g, 39.06 mmoles), and tetrakis(triphenylphosphine)palladium (800 mg, 6.92 mmoles) under Ar were added. The mixture was warmed overnight at 60° C. After cooling, the layers were separated and the organic layer was diluted with 2-methyl tetrahydrofuran (70 mL) and washed with sodium hydroxide solution (5%). The organic layer was concentrated in vacuo and the residue dissolved in toluene (150 mL) and 2-methyl tetrahydrofuran (50 mL). Thiol modified silicagel (1.3 g and Norit Supra A (5 g) were added and aged at room temperature for 2 h. After filtration and evaporation, the residue was crystallized from ethanol (90 mL) by slow addition of water (90 mL over 45 minutes). The mixture was aged overnight at room temperature, filtered and washed with ethanol:water (1:1). Yield: 7.6 g of intermediate 3 (78%) ((Z)-3-(4-(2-furyl)phenyl)-3-(3-thienyl)-2-propen-1-ol).

¹H NMR (600 MHz, CHLOROFORM-d) δ ppm 1.94 (br. s., 1H) 4.29 (d, J=6.80 Hz, 2H) 6.20 (t, J=6.80 Hz, 1H) 6.45 (dd, J=3.21, 1.70 Hz, 1H) 6.62 (d, J=3.02 Hz, 1H) 6.88 (dd, J=4.91, 1.13 Hz, 1H) 7.16 (dd, J=2.83, 1.32 Hz, 1H) 7.27 (dt, J=8.69, 1.89 Hz, 2H) 7.30 (dd, J=4.91, 3.02 Hz, 1H) 7.44 (d, J=1.51 Hz, 1H) 7.57 (dt, J=8.40, 2.03 Hz, 2H)

¹³CNMR (151 MHz, CHLOROFORM-d) δ ppm 60.52 (1 C) 105.18 (1 C) 111.67 (1 C) 123.50 (2 C) 124.71 (1 C) 125.26 (1 C) 127.76 (2 C) 127.97 (1 C) 129.03 (1 C) 130.11 (1 C) 138.56 (1 C) 138.88 (1 C) 140.39 (1 C) 142.12 (1 C) 153.60 (1 C)

### b. Preparation of Intermediate 3

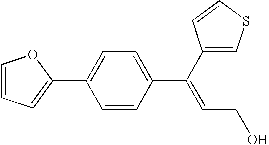

A round bottom flask was charged with intermediate 2 (0.50 g, 2.5 mmoles) and degassed tetrahydrofuran (3 mL) and cooled to 0° C. Sodium bis(2-methoxyethoxy)aluminum hydride (662.9 mg, 3.3 mmoles) was added and the mixture was stirred for 15 minutes. Ethyl acetate (88.9 mL, 1 mmole) was added to quench the reaction and 3-bromothiophene was added (neat, 534.6 mg, 3.3 mmoles). The reaction vessel was removed from the cooling bath and treated with zinc dichloride (2.4 g, 1.3 mmoles) and [1,3-bis(2,6-diisopropylphenyl)imidazol-2-ylidene](3-chloropyridyl palladium(II) dichloride (PEPPSI™ IPr) under an Ar blanket. The reaction mixture was quenched after 3 h at room temperature by addition of HCl (1 N) (10 mL) and extracted with 2-methyl tetrahydrofuran (10 mL). The organic layer was washed with sodium hydrogen carbonate, filtered and concentrated in vacuo. The residue was dissolved in dichloromethane and filtered through a pad of silica gel (30 g). The fractions containing the reaction product were collected and concentrated yielding crude (Z)-3-(4-(2-furyl)phenyl)-3-(3-thienyl)-prop-2-en-1-ol (490 mg, 69%; int.3). The product was further purified by dissolution in ethanol (10 mL), and precipitation by addition of water (8 mL). The precipitate was washed with ethanol:water (1:1, 5 mL), dried. Yield: 451 mg of intermediate 3 (92%) ((Z)-3-(4-(2-furyl)phenyl)-3-(3-thienyl)-2-propen-1-ol).

### B. Preparation of the Final Compounds

### Example B1

### a. Preparation of Compound 1

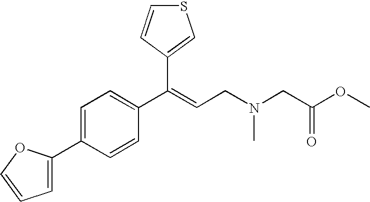

A solution of intermediate 3 (3.59 g, 12.4 mmoles) in dichloromethane (37.19 mL) was cooled to 0° C. Diisopropylethylamine (2.38 mL, 13.63 mmoles) was added. Methanesulfonyl chloride (1.01 mL, 13.02 mmoles) was added dropwise so that the temperature did not exceed 5° C. After stirring for 30 minutes at 0° C., sarcosine methyl ester hydrochloride (1.98 g, 13.63 mmoles) and diisopropylethylamine (4 .mL) were added while keeping the temperature below 4° C. The reaction mixture was stirred overnight protected from light by Al foil. The incomplete reaction was continued by addition of diisopropylethylamine (0.6 mL) and methanesulfonyl chloride (0.4 mL). The reaction was quenched with water. The organic layer was washed twice with water and then acidified with 1N HCl to pH 3. The aqueous layer basified with 1N NaOH and extracted with dichloromethane. The combined organic layers were concentrated. Yield: 4.95 g of compound 1 (methyl N-[(2Z)-3-[4-(2-furyl)phenyl]-3-(3-thienyl)-2-propen-1-yl]-N-methylglycinate) as a crude oil.

### b. Preparation of Compound 2

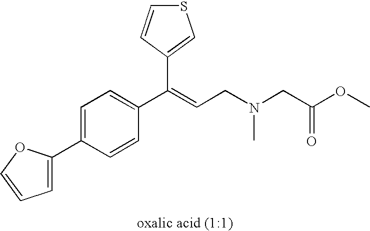

Compound 1 was dissolved in ethanol (27 mL), heated to 60° C. and treated with a solution of oxalic acid (1.48 g) in ethanol (18 mL). The precipitate was filtered and washed with ethanol (10 mL) affording methyl N-[(2Z)-3-[4-(2-furyl)phenyl]-3-(3-thienyl)-2-propen-1-yl]-N-methylglycinate compound with oxalic acid (1:1). Yield: 1.8 g of compound 2 (32%).

### Example B2

### a. Preparation of Compound 3

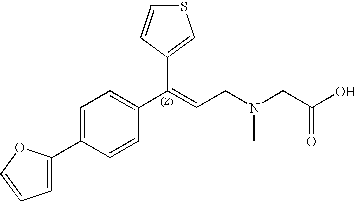

Compound 2 (20.2 g) was dissolved in methyl-tert.butyl ether (160 mL) and a solution of potassium carbonate (12.8 g) in water (160 mL) was added dropwise over 15 minutes. After stirring the mixture vigorously for 1 hour, the aqueous layer was removed and the organic layer was evaporated. The residue was dissolved in methanol (160 mL), treated with sodium hydroxide (3 mL 50%) and stirred at 60° C. for 4 hours. Water (120 mL and Norit Supra A (2 g) were added and stirring at 60° C. was continued for 30 minutes. The reaction mixture was filtered and reheated to 60° C. Formic acid (6.8 mL) was added dropwise over 15 minutes and stirring at 60° C. was continued for 90 minutes. After cooling to room temperature, the reaction mixture was stirred overnight. The precipitate was filtered of, washed with methanol:water (1:1, 100 ml), stirred for 1 hour in water (100 mL), filtered, washed with water and dried in vacuo at 50° C. Yield: 13.1 g of compound 3 (84%) (N-[(2Z)-3-[4-(2-furyl)phenyl]-3-(3-thienyl)-2-propen-1-yl]-N-methylglycine).

### b. Preparation of Compound 4

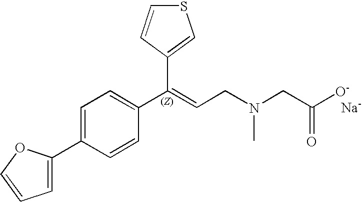

Compound 1 (7.5 g) was dissolved in methanol (80 mL) and sodium hydroxide (pellets, 0.88 g) was added. The mixture was heated at 60° C. for 2 hours. 2-Butanone (60 mL) was added and the temperature of the jacket was raised to 80° C. When about 50 mL solvent had been distilled of, 2-butanone (50 mL0 was added and the distillation of the solvent was continued. When 110 ml of distillate had been collected, 2-butanone (50 mL) was added and distillation was continued until the reactor temperature matched the jacket temperature (80° C.). A total of 173 mL solvent was removed. The reaction mixture was diluted with 2-butanone (25 mL), allowed to cool and filtered. The precipitate was washed with 2-butanone (30 mL) and dried in vacuo at 50° C. Yield: compound 4 (sodium N-[(2Z)-3-[4-(2-furyl)phenyl]-3-(3-thienyl)-2-propen-1-yl]-N-methylglycinate).

1. A compound having the formula (I) 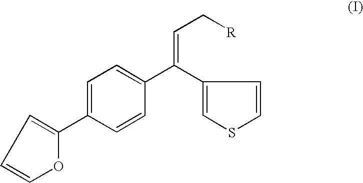
wherein R represents hydroxyl, or a leaving group L selected from the group consisting of chloro, bromo, iodo, methanesulfonyl, ethanesulfonyl, trifluoromethanesulfonyl, benzenesulfonyl, 4-methylbenzenesulfonyl, bromobenzenesulfonyl, and phosphonates.

2. The compound of claim 1 wherein R represents hydroxyl having the formula (I-a)

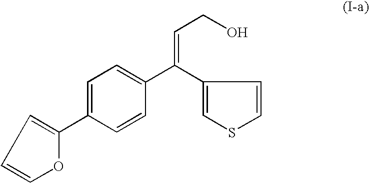

3. A process of converting a compound of formula (I-a) as defined in claim 2 into a compound of formula (II-a) or a pharmaceutically acceptable salt (II-b) thereof, 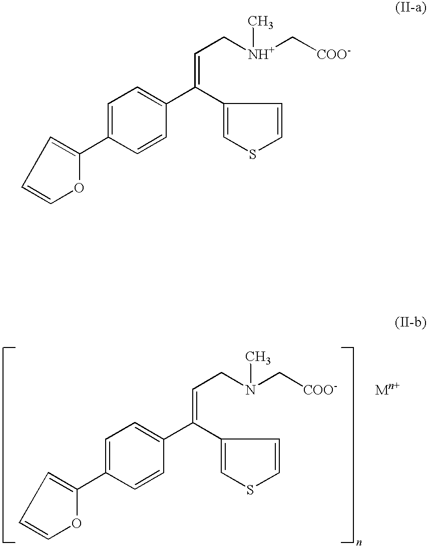
wherein n represents 1 or 2, and
Mⁿ⁺ represents a n-valent metal ion selected from the group consisting of the monovalent metal ions Li⁺, Na⁺, K⁺ and the divalent metal ions Mg²⁺ and Ca²⁺, comprising the steps of
(a) reacting the compound of (I-a) with a sulfonyl halide in a tertiary amine thus forming a compound of (I-b) 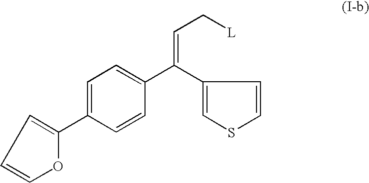
wherein L represents a leaving group as defined in claim 1;
(b) treating the compound of formula (I-b) in situ with sarcosine methyl ester hydrochloride thus forming a compound of formula (III) 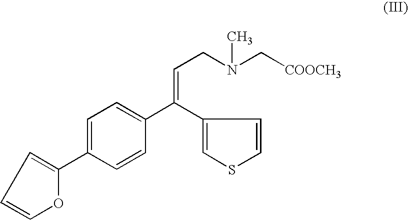
(c) hydrolyzing the compound of formula (III) in the presence of a base (Mⁿ⁺)(OH⁻)ₙ or (Mⁿ⁺)_{n/2} (CO₃²⁻) in a solvent to yield a compound of formula (II-b) 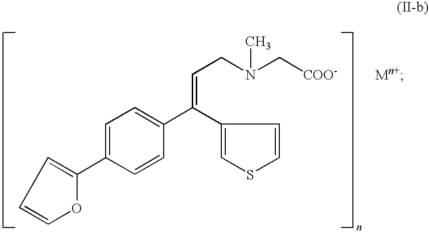
and
(d) optionally converting the thus obtained salt form (II-b) into the compound of formula (II-a) 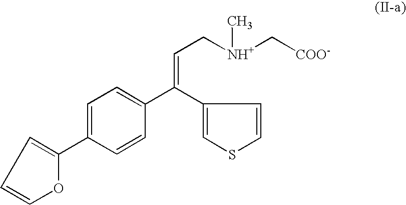
by treatment with an acid in an appropriate solvent.

4. A process of preparing the compound of formula (I-a) comprising the steps of
(a) coupling an intermediate of formula (IV) with 2-propyn-1-ol (V) in the presence of a Pd catalyst in an appropriate solvent to yield an intermediate of formula (VI) 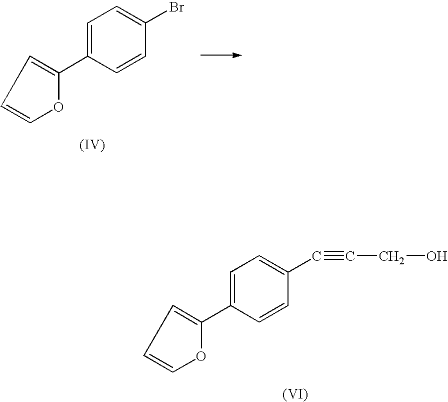
(b) converting the intermediate of formula (VI) by reaction with sodium bis(2-methoxyethoxy)aluminum hydride, followed by treatment in situ with iodine into an intermediate of formula (VII) 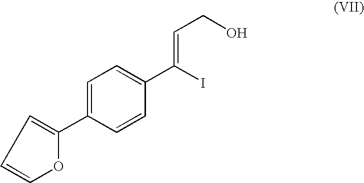
(c) coupling intermediate of formula (VII) with 3-thienylboronic acid (VIII) in the presence of Pd/C and triphenyl phosphine, thus yielding a compound of formula (I-a).

5. A process of preparing the compound of formula (I-a) comprising the steps of
(a) coupling an intermediate of formula (IV) with 2-propyn-1-ol (V) in the presence of a Pd catalyst in an appropriate solvent to yield an intermediate of formula (VI) 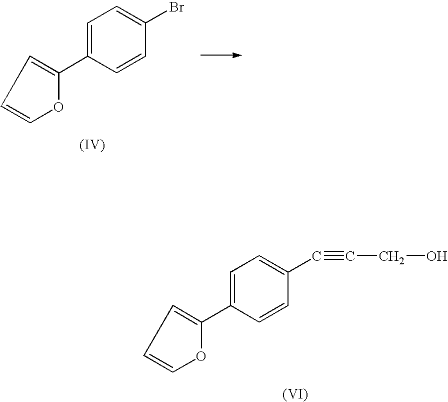
(b) converting the intermediate of formula (VI) by reaction with sodium bis(2-methoxyethoxy)aluminum hydride into an intermediate of formula (IX) 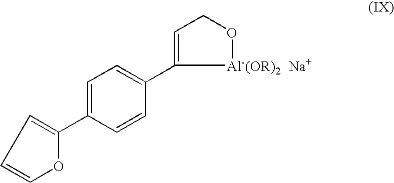
wherein each OR represents methoxyethoxy;
(c) coupling the intermediate of formula (IX) with 3-bromothiophene (X) in the presence of [1,3-bis(2,6-diisopropylphenyl)imidazol-2-ylidene](3-chloropyridyl palladium(II) dichloride (PEPPSI™ IPr) and zinc chloride, thus yielding a compound of formula (I-a).
